(19) United States
(12) Patent Application Publication
(10) Pub. No.: US 2024/0165189 A1
(43) Pub. Date: May 23, 2024

(54) COMPOSITION OF MATTER TO REDUCE BREAST LACTATION AND ENGORGEMENT

(71) Applicants: MONICA JACKSON, ATLANTA, GA (US); ARETHA HENDERSON, ATLANTA, GA (US)

(72) Inventors: MONICA JACKSON, ATLANTA, GA (US); ARETHA HENDERSON, ATLANTA, GA (US)

(21) Appl. No.: 18/086,817

(22) Filed: Dec. 22, 2022

Related U.S. Application Data
(60) Provisional application No. 63/378,942, filed on Nov. 18, 2022.

(51) Int. Cl. A61K 36/74; A61K 36/185; A61K 36/28; A61K 36/30; A61K 36/38; A61K 36/48; A61K 36/53; A61K 36/539; A61K 36/61; A61K 36/67; A61K 36/70; A61K 36/9066

(52) U.S. Cl. CPC A61K 36/74; A61K 36/185; A61K 36/28; A61K 36/30; A61K 36/38; A61K 36/48; A61K 36/53; A61K 36/539; A61K 36/61; A61K 36/67; A61K 36/70; A61K 36/9066

(57) ABSTRACT

The invention provides for efficacious compositions for extracting breast milk and relieving breast engorgement in lactating women. The composition is all natural and healthful for lactating women who breastfeed.

This non-provisional patent application claims priority to provisional patent application No. 63/378,942 filed on Nov. 18, 2022.

### FIELD OF THE INVENTION

The invention provides for efficacious compositions for extracting breast milk and relieving breast engorgement in lactating women. The composition is all natural and healthful for lactating women who breastfeed.

### BACKGROUND OF THE INVENTION

Breast engorgement is breast swelling that results in painful, tender breasts. It's caused by an increase in blood flow and milk supply in your breasts, and it occurs in the first days after childbirth.

Breast engorgement is the result of increased blood flow in a woman's breasts in the days alter the delivery of a baby. The increased blood flow helps your breasts make ample milk, but it can also cause pain and discomfort. Milk production may not occur until three to five days postpartum. Engorgement may occur for the first time in the first week or two after delivery. It can also reoccur at any point if you continue to breastfeed.

Certain conditions or events may make you more likely to experience the swollen fullness that's commonly associated with breast engorgement. These causes may include but are not limited to missing a feeding, skipping a pumping session, creating an overabundance of milk for the baby's appetite, supplementing with formula between nursing sessions, which may reduce nursing later, weaning too quickly, nursing a baby that's ill, difficulty with latching and sucking, and/or not expressing breast milk when it first comes in because you don't plan to breastfeed.

What is needed and what is provided herein is a useful composition to reduce lactation and breast engorgement in would-be breast feeding women.

### SUMMARY OF THE INVENTION

Accordingly, the invention provides A composition of matter for postpartum women to extract breast milk to reduce lactation and breast engorgement, comprising:
a. Coffee in about 0.01-1 part by weight;
b. Lavender in about 0.01-1 part weight:
c. Hop flower in about 0.01-1 part by weight;
d. Cloves in about 0.01-1 part by weight;
e. Yellow Dock in about 0.01-1 part by weight
f. Yarrow Flowers in about 0.01-1 part by weight;

g. St. John's Wort in about 0.01-1 part by weight;

h. Red Clover in about 0.01-1 part by weight;

i. Passionflower in about 0.01-1 part by weight;

j. Kava in about 0.01-1 part by weight;

k. Nettle Stinging in about 0.01-1 part by weight;
l. Skullcaps in about 0.01-1 part by weight;
m. Chamomile in about 0.01-1 part by weight;
n. Burdock in about 0.01-1 part by weight;
o. Turmeric in about 0.01-1 part by weight;
p. Comfrey leaf in about 0.01-1 part by weight; and
q. Damiana in about 0.01-1 part by weight.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### DETAILED DESCRIPTION OF THE INVENTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “may,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods.

Thus, if there are a variety of additional steps that can be performed it is understood that each of these additional steps can be performed with any specific aspect or combination of aspects of the disclosed methods.

The invention herein provides a composition of matter for postpartum women to extract breast milk to reduce lactation and breast engorgement, comprising:
a. Coffee in about 0.01-1 part by weight;
b. Lavender in about 0.01-1 part weight:
c. Hop flower in about 0.01-1 part by weight;
d. Cloves in about 0.01-1 part by weight;
e. Yellow Dock in about 0.01-1 part by weight
f. Yarrow Flowers in about 0.01-1 part by weight;
g. St. John's Wort in about 0.01-1 part by weight;
h. Red Clover in about 0.01-1 part by weight;
i. Passionflower in about 0.01-1 part by weight;
j. Kava in about 0.01-1 part by weight;
k. Nettle Stinging in about 0.01-1 part by weight;
l. Skullcaps in about 0.01-1 part by weight;
m. Chamomile in about 0.01-1 part by weight;
n. Burdock in about 0.01-1 part by weight;
o. Turmeric in about 0.01-1 part by weight;
p. Comfrey leaf in about 0.01-1 part by weight; and
q. Damiana in about 0.01-1 part by weight.

The primary composition herein contains several ingredients listed and described hereinbelow. Each ingredient is important and operates in combination with all other included ingredients to achieve the efficacious results sought.

Coffee described herein is a beverage brewed from the roasted and ground seeds of the tropical evergreen coffee plant. The main constituents of coffee are caffeine, tannin, fixed oil, carbohydrates, and proteins. It contains 2-3% caffeine, 3-5% tannins, 13% proteins, and 10-15% fixed oils. In the seeds, caffeine is present as a salt of chlorogenic acid.

The lavender described herein comes from lavender plants which are small, branching and spreading shrubs with grey-green leaves and long flowering shoots. The leaves can be simple or pinnate measuring 30-50 mm (1-2 in) in length. The plant produces flowers on shoots or spikes which can be 20-40 cm (8-16 in) long. The flowers are lilac or blue in color. The main chemical compounds thereof are linalool (39%), α-terpineol (15%), and coumarin (7%). The hydrolates lack in linalyl acetate, a chemical compound present in a large amounts (1.2%-59.4%) in the essential oil of lavender.

The hop flower used in the chemical composition herein derives from the hop plant, either of two species of the genus Humulus, nonwoody annual or perennial vines in the hemp family (Cannabinaceae) native to temperate North America, Eurasia, and South America. The hop flower contains myrcene, humulene, xanthohumol, myrcenol, linalool, tannins, and resin. Hops are unique for containing secondary metabolites, flavonoids, oils, and polyphenols that impact the flavor of the products they are common in, such as beer.

Cloves used herein vary in length from about 13 to 19 mm (0.5 to 0.75 inch). The buds contain 14 to 20 percent essential oil, the principal component of which is the aromatic oil eugenol. Cloves are strongly pungent owing to eugenol, which is extracted by distillation to yield oil of cloves. The main components there are eugenol (76.8%), followed by β-caryophyllene (17.4%), α-humulene (2.1%), and eugenyl acetate (1.2%).

Yellow dock or yellow dock root (Rumex crispus) is a common weed found in Serbia, Korea, and China. The leaf stalks are used in salads. The root and fruits are used as medicine, Yellow dock contains chemicals that work as laxatives and have anti-inflammatory effects. Yellow dock root contains: 2-4% anthraquinones including chrysophanol, emodin, nepodin and physcion (aglycones). Tannins such as Catechol (5%) (condensed-type). Other plant constituents documented include oxalic acid, oxalates, chrysophanic acid and a complex volatile oil.

Yarrow is a hardy perennial plant, native to Europe and Asia. The chemical constituents include a volatile oil comprised of azulene, caryophyllene, thujone, eucalyptol, α- and β-pinene and borneol, as well as lactones, tannins, and alkaloids. Popular in European folk medicine, yarrow contains flavonoids, plant-based chemicals that increase saliva and stomach acid to help improve digestion. Yarrow may also relax smooth muscle in the intestine and uterus, which can relieve stomach and menstrual cramps.

John's wort contains several chemicals, including hypericin, hyperforin, and flavonoids. Researchers aren't exactly sure how St. John's wort works. Some have suggested that the herb acts similar to an SSRI, increasing the availability of the brain chemicals serotonin, dopamine, and norepinephrine. John's Wort acts as a serotonin reuptake inhibitor. This works to reduce the uptake of serotonin at neuronal synapses, as well as dopamine and norepinephrine. Elevated levels of neurotransmitters are believed to be helpful when treating depression.

Red clover's chemical name is known as Trifolium pratense fabales. Red clover (Trifolium pratense) is an herb that belongs to the legume family, along with beans and peas. Red clover benefits are thought to arise from the many nutrients found in its flowers, including calcium, chromium, magnesium, niacin, phosphorus, potassium, thiamine, and vitamin C. Chemically, red clover comprises 35.54% isoflavones, 1.11% flavonoids, 0.06% pterocarpans, ≤10.03% coumarins, and ≤0.03% tyramine.

Passionflower (i.e., Passiflora incarnata) was used traditionally in the Americas and later in Europe as a calming herb for anxiety, insomnia, seizures, and hysteria. It is still used today to treat anxiety and insomnia. Chemically it contains several flavonoids (apigenin, benzoflavone and others), harmala alkaloids (hamaline, harmalol, harmine and harmol), coumarins, maltol, phytosterols and glycosides.

Kava, also spelled cava or ava, nonalcoholic euphoria-producing beverage made from the root of the pepper plant, principally Piper methysticum, in most of the South Pacific islands. Chemically, kava comprises three chemical classes: (i) arylethylene-alpha-pyrones; (ii) chalcones and other flavanones; and (iii) conjugated diene ketones. The substituted 4-methoxy-5, 6-dihydro-alpha-pyrones or kava pyrones, commonly called kavalactones, possesses the highest purported pharmacological activities.

Stinging (hair of) nettle has antiproliferative, anti-inflammatory, antioxidant, analgesic, anti-infectious, hypotensive, and antiulcer characteristics, as well as the ability to prevent cardiovascular disease, in all parts of the plant leaves, stems, roots, and seeds). Chemically, it contains formic acid, histamine, and acetylcholine. Leaves of nettle contain provitamin A, vitamin B₁, K, xanthophylls, and sistosterin and ashes of nettle contain 6.3% ferric oxide, potassium, calcium, and silicium.

Skullcap can refer to 2 herbs: American skullcap (Scutellaria lateriflora) and Chinese skullcap (Scutellaria baicalensis). Skullcap is a flowering plant long used in traditional medicine. Supplementing with skullcap may provide several health benefits, such as improved mood, reduced inflammation, and anticancer effects. The main compounds in skullcap are flavonoids. Isolated chemical compounds include wogonin, wogonoside, and 3,5,7,2′,6′-pentahydroxyl flavanone found in Scutellaria. Other constituents include baicalin, apigenin, oroxylin A, and scutellarein.

The best-known botanical name for true chamomile is Matricaria recutita (syn, Matricaria chamomilla, Chamomilla recutita (L.) Rauscher, belonging to the genus Chamomilla and family Asteraceae. Chamomile has been valued as a digestive relaxant and has been used to treat various gastrointestinal disturbances including flatulence, indigestion, diarrhea, anorexia, motion sickness, nausea, and vomiting. Chamomile has also been used to treat colic, croup, and fevers in children. The flowers of chamomile contain 1-2% volatile oils including alpha-bisabolol, alpha-bisabolol oxides A & B, and matricin (usually converted to chamazulene and other flavonoids which possess anti-inflammatory and antiphlogistic properties.

Arctium lappa L. (Asteraceae), also known as “burdock,” is a perennial and cultivated plant used as a vegetable in many countries. In Traditional Chinese Medicine (TCM), burdock is often used with other herbs for sore throat and colds. In Japan and some parts of Europe, burdock is eaten as a vegetable. Burdock contains inulin, a natural dietary fiber, and is also used to improve digestion. Chemically, fresh burdock root is chemically composed of ca. 70% water, 2.8% protein, 25% carbohydrate and 0.6% ash. Burdock root contains mostly inulin, amino acids, sulphur-acetylene class, multi-polyacetylenes, poly-phenols and volatile oil, among others.

Tumeric is a diferuloylmethane with a crystalline yellow-orange colour, molecular weight of 368.39 g/mol, melting temperature of 183° C., and with the chemical formula C₂₁H₂₀O₆. Turmeric and especially its most active compound, curcumin have many scientifically proven health benefits, such as the potential to improve heart health and prevent against Alzheimer's and cancer. It's a potent anti-inflammatory and antioxidant. It may also help improve symptoms of depression and arthritis.

Comfrey is a perennial shrub that is native to Europe and some parts of Asia. Fond of moist soils, comfrey has a thick, hairy stem, and grows 2 to 5 feet tall. Comfrey contains substances that help skin regrow, including allantoin, rosmarinic acid, and tannins. It also has poisonous chemicals called pyrrolizidine alkaloids. Comfrey roots and leaves contain allantoin, a substance that helps new skin cells grow, along with other substances that reduce inflammation and keep skin healthy. Comfrey ointments have been used to heal bruises as well as pulled muscles and ligaments, fractures, sprains, strains, and osteoarthritis.

Damiana's relaxing and uplifting qualities can be useful for anxiety, mild depression, and nervous exhaustion. Its combined influence on the nervous and reproductive system make it especially useful for depression and anxiety associated with sexual issues. Damiana contains damianin; tetraphyllin B; gonzalitosin I; arbutin; tricosan-2-one; acacetin; p-cymene; β-sitosterol; 1,8-cineole; apigenin; α-pinene; β-carotene β-pinene; tannins; thymol; and hexacosanol.

All of the materials and ingredients noted herein may be decomposed (i.e., broken into finer parts) and mixed in a standard mixer of the kinds well known by persons of skill in the art. The mixture is then consumable either orally or intravenously by a user as appropriate at the recommendation of a health care professional.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter for postpartum women to extract breast milk to reduce lactation and breast engorgement, comprising:
a. Coffee in about 0.01-1 part by weight;
b. Lavender in about 0.01-1 part weight:
c. Hop flower in about 0.01-1 part by weight;
d. Cloves in about 0.01-1 part by weight;
e. Yellow Dock in about 0.01-1 part by weight
f. Yarrow Flowers in about 0.01-1 part by weight;
g. St. John's Wort in about 0.01-1 part by weight;
h. Red Clover in about 0.01-1 part by weight;
i. Passionflower in about 0.01-1 part by weight;
j. Kava in about 0.01-1 part by weight;
k. Nettle Stinging in about 0.01-1 part by weight;
l. Skullcaps in about 0.01-1 part by weight;
m. Chamomile in about 0.01-1 part by weight;
n. Burdock in about 0.01-1 part by weight;
o. Turmeric in about 0.01-1 part by weight;
p. Comfrey leaf in about 0.01-1 part by weight; and
q. Damiana in about 0.01-1 part by weight.
